(19) United States
(12) Patent Application Publication
(10) Pub. No.: US 2021/0369881 A1
(43) Pub. Date: Dec. 2, 2021

(54) LOW-FLOW HAND SANITATION UNIT

(71) Applicant: Rheem Manufacturing Company, Atlanta, GA (US)

(72) Inventors: Christopher M. Hayden, Shelton, CT (US); Harsha Satyanarayana, Bristol, CT (US); Bill Dahlin, Waterbury, CT (US)

(21) Appl. No.: 16/890,275

(22) Filed: Jun. 2, 2020

(51) Int. Cl. A61L 2/00; A61L 2/24

(52) U.S. Cl. CPC A61L 2/0094; A61L 2/24; A61L 2202/11; A61L 2202/14; A61L 2202/15; A61L 2/0047

(57) ABSTRACT

The disclosed technology includes a system for sanitizing a user's hands or other objects using atomized water. The system can include a heating element for heating air, a blower for directing the heated air toward a user's hands or other objects, and one or more reservoirs for holding fluids, such as water and a sanitizing agent. The system can include an atomizing device for atomizing at least some of the fluid. The system can include a controller for controlling the various components of the system.

### FIELD OF TECHNOLOGY

The present disclosure relates generally to systems that can be used to sanitize a user's hands, and, more particularly, to sanitizing systems that utilize atomized fluids to sanitize a user's hands.

### BACKGROUND

Germs (e.g., bacteria, viruses, fungi, protozoa, or other microorganisms that cause disease) can be easily transferred from one individual to another by touching contaminated surfaces or by shaking hands, making hand sanitation a vital aspect of promoting human health. One of the most common and effective methods of sanitizing hands includes cleaning with soap and water to remove germs from the hands. Soap and water are often located at a sink in restrooms, kitchens, or other locations where germs are likely to accumulate, thereby providing easy access to the soap and water to sanitize their hands. Using soap and water, however, is not always practical. For example, soap and water may not be readily available in locations without plumbing or may be impractical when large crowds are gathered. The inconvenience of washing hands with soap and water can lead some individuals to forego hand washing altogether.

Because using soap and water is not practical in every situation, many individuals use chemical solutions, such as alcohol or benzalkonium chloride-based hand sanitizers, to sanitize their hands. Hand sanitizer dispensers can be easily installed in various locations because plumbing is not required. Furthermore, hand sanitizers can even be portable, such as in a bottle or other package, so that a user can carry a small supply of hand sanitizer to sanitize his or her hands in almost any location. These hand sanitizers, however, can provide an unpleasant experience for many users because the chemical solutions can emit a strong odor and repeated use of the hand sanitizers can lead to dry skin or other uncomfortable skin irritations which can further discourage individuals from sanitizing their hands. Such ill effects can be particularly prevalent for individuals who must frequently sanitize their hands, such as food workers, medical care professionals, and others.

What is needed, therefore, is a readily-accessible sanitizing system that provides hand sanitation without the need for piped plumbing systems or chemicals, while reducing or eliminating skin irritation or discomfort. This and other problems are addressed by the technology disclosed herein.

### SUMMARY

The disclosed technology relates generally to systems that can be used to sanitize a user's hands or other objects, and, more particularly, to sanitizing systems that utilize atomized fluids to sanitize a user's hands or other objects.

The disclosed technology can include a hand-sanitizing system having a heating element for heating air and a blower to direct the heated air toward a user's hands or other object. The system can include one or more reservoirs to hold fluids such as water and/or a sanitizing agent. The system can also include an atomizing device configured to atomize at least one of the fluids and a controller configured to activate the blower, the heating element, and the atomizing device to direct heated air and the fluids toward a user's hands or other object.

The system can include an ultraviolet (UV) light that can be controlled by the controller to disinfect a user's hands or other object. The UV light can be a Far Ultraviolet-C (Far UV-C) light source.

Alternatively, the blower can be configured to create a venturi effect to direct the fluids toward the user's hands or other object.

The system can also include a proximity sensor that can detect the presence of the user's hand. The controller can activate the blower and the heating element in response to determining that the user's hand is within a predetermined distance from the proximity sensor based on data received from the proximity sensor. The controller can also deactivate the blower and the heating element if the presence of a user is no longer detected.

The heating element can additionally heat the fluids. Furthermore, the fluids can be atomized using an ultrasonic transducer or compressed air. If the fluid is a sanitizing agent, the sanitizing agent can be an antibacterial solution or electrolyzed water.

The system can include a collection system to collect overspray of the fluids after the fluids are directed toward the user's hands or other object. The collection system can include a vacuum and an accumulation tank. The vacuum can be created by the blower or it can be a standalone system.

The system can include a pump configured to transport at least some of the fluids or the system can include flow control valves to control the flow of the fluids. Optionally, the system can be powered by a battery and a solar power system configured to charge the battery.

Additional features, functionalities, and applications of the disclosed technology are discussed herein in more detail.

### BRIEF DESCRIPTION OF THE DRAWINGS

The accompanying drawings, which are incorporated in and constitute a part of this specification, illustrate multiple examples of the presently disclosed subject matter and serve to explain the principles of the presently disclosed subject matter. The drawings are not intended to limit the scope of the presently disclosed subject matter in any manner.

FIG. 1 is a schematic diagram illustrating a system for sanitizing hands, in accordance with an example of the disclosed technology.

FIG. 2 is a schematic diagram illustrating another system for sanitizing hands, in accordance with an example of the disclosed technology.

FIG. 3 is flowchart illustrating a method of sanitizing hands using a sanitizing system, in accordance with an example of the disclosed technology.

### DETAILED DESCRIPTION

The disclosed technology relates generally to sanitizing systems that utilize atomized fluids to sanitize a user's hands or other objects. The disclosed technology includes a hand sanitizing device or unit that can be mounted on a wall or a pedestal. As will be understood in view of the disclosure herein, the disclosed technology includes systems and devices for sanitizing hands or other objects without the need for a plumbing system. Rather than using a plumbing system, the disclosed technology can include a fluid reservoir and dispense ultra-low quantities of fluid by atomizing the fluid as it is directed toward the user. The fluid can include water and an antibacterial agent and can be stored together in a single fluid reservoir or can be stored in separate fluid reservoirs. Because the disclosed technology can dispense ultra-low flows of the fluid, certain systems and devices in accordance with the disclosed technology can be used without a corresponding drain system. That is, the atomized fluid can be dried on a user's hands. The drying process can be expedited in some instances by simply directing air across the user's hands using a blower. Furthermore, systems and devices in accordance with the disclosed technology can incorporate a heating element to heat the air and/or water to provide for a more pleasing user experience and to further sanitize the user's hands. Systems and devices in accordance with the disclosed technology can additionally include an ultraviolet light source configured to emit an ultraviolet light to provide an additional method for disinfecting the user's hands.

Although certain examples of the disclosed technology are explained in detail herein, it is to be understood that other examples, embodiments, and implementations of the disclosed technology are contemplated. Accordingly, it is not intended that the disclosed technology is limited in its scope to the details of construction and arrangement of components expressly set forth in the following description or illustrated in the drawings. The disclosed technology can be implemented in a variety of examples and can be practiced or carried out in various ways. In particular, the presently disclosed subject matter is described in the context of providing a sanitizing system that utilizes atomized fluids to sanitize a user's hands. The present disclosure, however, is not so limited, and can be applicable in other contexts where sanitation is desirable. The present disclosure, for example and not limitation, can include systems for sanitizing objects such as packages, medical devices, cooking utensils, etc., and surfaces known to accumulate germs such as countertops, door handles, elevator buttons, and other surfaces that are commonly touched. Such implementations and applications are contemplated within the scope of the present disclosure. Accordingly, when the present disclosure is described in the context of being a sanitizing system that utilizes atomized fluids to sanitize a user's hands, it will be understood that other implementations can take the place of those referred to.

It should also be noted that, as used in the specification and the appended claims, the singular forms “a,” “an,” and “the” include plural references unless the context clearly dictates otherwise. References to a composition containing “a” constituent is intended to include other constituents in addition to the one named.

Also, in describing the examples, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” or “substantially” one particular value and/or to “about” or “approximately” or “substantially” another particular value. When such a range is expressed, the various examples of the disclosed technology includes from the one particular value and/or to the other particular value. Further, ranges described as being between a first value and a second value are inclusive of the first and second values. Likewise, ranges described as being from a first value and to a second value are inclusive of the first and second values.

Herein, the use of terms such as “having,” “has,” “including,” or “includes” are open-ended and are intended to have the same meaning as terms such as “comprising” or “comprises” and not preclude the presence of other structure, material, or acts. Similarly, though the use of terms such as “can” or “may” are intended to be open-ended and to reflect that structure, material, or acts are not necessary, the failure to use such terms is not intended to reflect that structure, material, or acts are essential. To the extent that structure, material, or acts are presently considered to be essential, they are identified as such.

It is also to be understood that the mention of one or more method steps does not preclude the presence of additional method steps or intervening method steps between those steps expressly identified. Moreover, although the term “step” can be used herein to connote different aspects of methods employed, the term should not be interpreted as implying any particular order among or between various steps herein disclosed unless and except when the order of individual steps is explicitly required. Further, the disclosed technology does not necessarily require all steps included in the example methods and processes described herein. That is, the disclosed technology includes methods that omit one or more steps expressly discussed with respect to the examples provided herein.

The components described hereinafter as making up various elements of the disclosed technology are intended to be illustrative and not restrictive. Many suitable components that would perform the same or similar functions as the components described herein are intended to be embraced within the scope of the disclosed technology. Such other components not described herein can include, but are not limited to, for example, similar components that are developed after development of the presently disclosed subject matter.

Referring now to the drawings, in which like numerals represent like elements, examples of the present disclosure are herein described. As will be described in greater detail, the present disclosure can include a sanitizing system that utilizes atomized fluids to sanitize a user's hands.

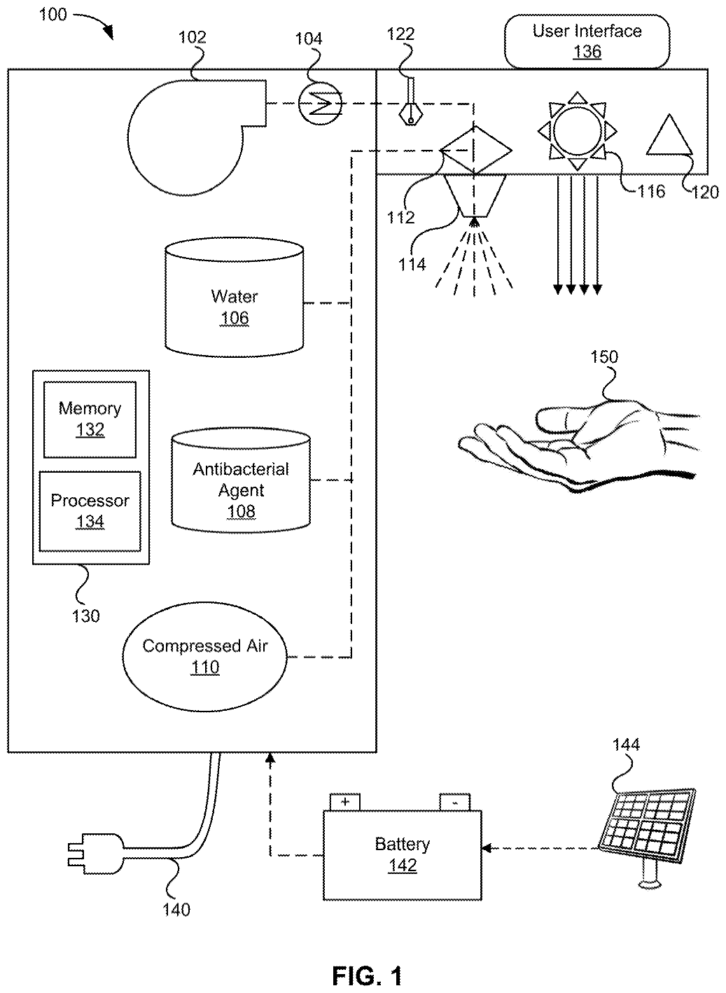

As depicted in FIG. 1, the sanitizing system 100 can be a device configured to provide a means whereby a user 150 can wash his or her hands. The sanitizing system 100 can be mountable (e.g., on a wall) or installed on a stand or pedestal (e.g., a rollable or otherwise transportable pedestal) so that a user 150 can approach the sanitizing system 100 to sanitize his or her hands in locations where sinks or other hand washing stations requiring plumbing are unavailable (or to supplement hand sanitation afforded by existing sinks or other hand washing stations). To provide an overview of the sanitizing system 100, the user 150 can approach the sanitizing system 100 and place his or her hands beneath a dispenser 114. The sanitizing system 100 can then activate a blower 102 and a heating element 104 and begin directing atomized water from a water reservoir 106 to the user's 150 hands. The mixture of heated air and atomized water results in the user 150 being able to rinse his or her hands with heated water. The sanitizing system 100 can additionally direct an antibacterial agent (e.g., an antibacterial solution) from an antibacterial agent reservoir 108 toward a user's hands to sanitize the user's 150 hands. The water from the water reservoir 106 and/or the antibacterial agent from the antibacterial agent reservoir 108 can be atomized using an atomizing device 112 so that the user 150 can wash his or her hands using small amounts of the water and the antibacterial solution. The water and antibacterial agent can be individually atomized with the atomizing device 112 prior to being directed toward the user's 150 hands. Alternatively, the water and antibacterial agent can be mixed together prior to being atomized with the atomizing device 112. The sanitizing system 100 can also include an ultraviolet light source 120 that is configured to disinfect the user's 150 hands. Further features and details of the sanitizing system will be described herein.

The blower 102 can be configured to direct air toward the user's 150 hands when activated. The blower 102 can be used in conjunction with a heating element 104 that is configured to heat the air being moved by the blower 102. As one of skill in the art will appreciate, when water or other fluid is atomized by the atomizing device 112, heat retained by the water or other fluid can quickly dissipate leaving the user 150 with cool fluid to wash his or her hands. As many users 150 are accustomed to using warm water to sanitize his or her hands, delivering the atomized fluid with a supply of warm air can provide the user 150 with a perceived warmth of the atomized fluid, which can result in a more pleasing user experience for the user 150. Furthermore, utilizing heated air to direct water or other fluid toward the user's 150 hands can help to further disinfect the user's 150 hands because certain germs (e.g., bacteria, viruses) can be weakened or destroyed by heat above a certain temperature. Accordingly, heated air can increase the effectiveness of disinfecting hands or other surfaces.

The blower 102 can be any type of blower suitable for the application. The blower 102, for example, can be a positive displacement blower, a rotary lobe blower, a helical screw blower, a centrifugal blower, a high-speed blower, a regenerative blower, or any other type of blower suitable for the application. The blower 102 can be a single-speed blower or the blower 102 can be a multi-stage blower configured to direct air at more than one speed. Sanitizing systems 100 that comprise a multi-stage blower can be used to rinse the user's 150 hands by directing heated air and atomized fluid at low speeds. Multi-stage blowers can also be used to dry the user's 150 hands by directing heated air at high speeds. Systems that do not have a multi-stage blower can have more than one blower 102 where one blower is configured to operate at low speeds to direct the heated air and fluid to the user's hands while a second blower is configured to operate at high speeds to dry the user's hands. To further increase the effectiveness of the blower 102 when used to dry a user's 150 hands, the blower 102 can be used with an air knife or other device configured to increase the velocity of the air as it is directed from the blower. As one of skill in the art will appreciate, using high velocity air can shorten the time needed to dry the user's hands.

The heating element 104 can be any type of heating element suitable for the application. The heating element 104, for example, can be an electrical heating element, a propane burner, a natural gas burner, or any other suitable heating element for the application. The heating element 104 can be installed upstream of the blower's 102 intake, inside a portion of the blower 102, or anywhere downstream of the blower 102 so that the air is heated prior to reaching the user's 150 hands. Alternatively, or in addition, the heating element 104 (or a separate heating element) can be configured to heat the water or antibacterial solution. The heating element 104 (or a separate heating element) can be configured to heat the water and/or antibacterial agent while it is stored in the water reservoir 106 or antibacterial agent reservoir 108, respectively. Alternatively, or in addition, the heating element 104 (or a separate heating element) can be configured to heat the water and/or antibacterial agent as it is being transported or directed toward the user's 150 hands.

As will be discussed in greater detail herein, the heating element 104 can be controlled by a controller 130. The controller 130 can be configured to receive an input from a temperature sensor 122 configured to detect a temperature of the air as it is directed toward the user 150 by the blower 102. Based on the input received, the controller 130 can vary the output of the heating element to ensure the air is heated to a target temperature. The target temperature can be a predetermined temperature value, a predetermined range of temperature values, a user-inputted temperature value, or a user-inputted range of temperature values. The temperature sensor 122 can include any type of sensor capable of measuring the temperature of the air as it is directed from the blower 102 toward the user 150. For example, the temperature sensor 122 can be or include a thermocouple, a resistance temperature detector (RTD), a thermistor, an infrared sensor, a semiconductor, or any other suitable type of sensor.

Because the sanitizing system 100 is configured to direct small amounts of atomized fluid toward the user's 150 hands, the sanitizing system 100 does not require a plumbing system because the amounts of fluid directed toward the user's 150 hands are reduced to the level where a large source of fluid is unnecessary. As one of skill in the art will appreciate, unlike traditional hand washing systems, a sanitizing system 100 configured to direct atomized fluid at ultra-low flow rates of about 0.01 gallons per minute to about 0.1 gallons per minute does not require a large amount of available fluid. Furthermore, the small amount of water or other fluid directed toward the user's 150 hands are reduced to the level where excess water or other fluid is unlikely to flow past the user's hands. Therefore, the sanitizing system 100 does not require a drain like traditional hand washing systems. Because of this, the sanitizing system 100 can have a water reservoir 106 configured to store a sufficient amount of water for the application and can direct the fluid toward the user's 150 hands without needing to collect fluid that escapes past the user's 150 hands. For example, if the sanitizing system 100 is to be installed on an individual pedestal in the middle of a grocery store or other public location, the sanitizing system 100 can have a water reservoir 106 configured to store an amount of water in the range from about 0.5 gallons to about 5 gallons, depending on the application, and does not require a drain to collect fluid that has escaped past the user's 150 hands. Of course, the system 100 can include a water reservoir 106 of any size, depending on the application.

Alternatively, the sanitizing system 100 can include a water reservoir 106 that comprises a plumbing system, such as a traditional plumbing system from a large water source but is configured to direct minimal amounts of water when demanded. In this configuration, the sanitizing system 100 can still direct minimal amounts of water but does not require a user 150 to replace the water reservoir 106 when emptied because the plumbing system will continue to deliver water to the sanitizing system 100.

The water from the water reservoir 106 can be directed toward a user's 150 hands using any method suitable for the application. The water from the water reservoir 106, for example, can be directed toward the user's 150 hands by using a pump, a valve where the fluid is pressurized or gravity fed, or even a system intended to create a negative pressure with the blower 102 (e.g., a venturi system). No matter the preferred system, one of skill in the art will appreciate that the water from the water reservoir 106 can be directed toward the user's hands at suitable flow rates to adequately facilitate hand sanitization while reducing the overall amount of water used.

The sanitizing system 100 can include an antibacterial agent reservoir 108 that is configured to store an antibacterial solution. The antibacterial agent reservoir 108 can be configured to store any type of antibacterial agent including, but not limited to, antibacterial solutions containing alcohol, chlorine, peroxide, aldehyde, triclosan, triclocarban, benzalkonium chloride, or antibacterial soaps, regular soaps, electrolyze water, or any other substance that can be used to kill or remove germs from the surface of a user's 150 hands. The antibacterial agent in the antibacterial agent reservoir 108 can be in liquid, gel, or solid form as would be suitable for the particular application. Furthermore, more than one antibacterial agent reservoir 108 can be used if it is desirable to use more than one antibacterial agent or to combine two or more parts antibacterial agent prior to directing the antibacterial agent to the user 150. Like the water reservoir 106, the antibacterial reservoir 108 can be any designed volume, depending on the application. The antibacterial agent can include a moisturizing solution configured to moisturize a user's 150 hands. Alternatively, the sanitizing system 100 can include a separate reservoir for storing a moisturizing solution.

The antibacterial agent from the antibacterial agent reservoir 108 can be directed toward a user's 150 hands using any method suitable for the application. The antibacterial agent from the antibacterial agent reservoir 108, for example, can be directed toward the user's 150 hands by using a pump, a valve where the fluid is pressurized or gravity fed, or even a system intended to create a negative pressure with the blower 102 (e.g., a venturi system) to direct the antibacterial agent toward the user's 150 hands. No matter the preferred system, one of skill in the art will appreciate that the antibacterial agent from the antibacterial agent reservoir 108 can be directed toward the user's hands at suitable flow rates to adequately facilitate hand sanitization while reducing the overall amount of antibacterial agent used.

The sanitizing system 100 can include a compressed air source 110 that is configured to provide compressed air for directing the fluid to the user 150. A compressed air source 110 can be useful, for example, if the sanitizing system 100 is configured to atomize the fluid using compressed air and a dispenser 114 that is a nozzle configured to atomize the fluid. As will be appreciated by one of skill in the art, the compressed air source 110 can be used to pressurize the fluid so that the fluid can be atomized through the nozzle and directed toward the user 150. The compressed air source 110 can be an air compressor, an aerosol mixture, a compressed air tank, or any other suitable compressed air source for the application.

Alternatively, the sanitizing system 100 can include an ultrasonic atomizer 112 that is configured to atomize the fluid as is it directed toward the user 150. The ultrasonic atomizer 112 can be used in combination with the compressed air source 110, or the ultrasonic atomizer 112 can be used without the need for the compressed air source 110. As an example, the ultrasonic atomizer 112 can be an ultrasonic nozzle built into the dispenser 114 having a piezoelectric transducer configured to atomize the fluid. As one of skill in the art will appreciate, the ultrasonic atomizer 112 can atomize the fluid at low flows which can reduce the total amount of fluid used, making the sanitizing system 100 require less fluid storage and remove the need for a drain system. The ultrasonic atomizer 112 can be configured to direct the atomized fluid to the user 150 or can be configured to direct the atomized fluid into the path of the heated air directed by the blower 102 toward the user.

The sanitizing system 100 can alternatively use other methods of atomizing the fluid including a rotary atomizer, a twin-fluid atomizer, an electrostatic atomizer, or any other suitable method of atomizing the fluid. As one of skill in the art will appreciate, the chosen method of atomizing the fluid can be capable of atomizing fluids at low flows such that the overall amount of fluid used is reduced.

The sanitizing system 100 can have an ultraviolet (UV) light source 116 configured to direct UV light toward a user's 150 hand to further disinfect the surface of the user's 150 hands. The UV light source 116 can be any form of UV light source capable of disinfecting a surface, however, as one of skill in the art will appreciate, many types of UV light can be harmful for human use. Therefore, the sanitizing system 100 can use a UV light source 116 that is less likely to harm a user 150 such as a Far UV-C light source or other UV light sources that are less likely to harm a user. The UV light source 116, for example, can be configured to have a wavelength of about 207-222 nanometers such that the UV light is unlikely to penetrate a user's skin or eyes but is able to kill germs. The UV light source 116 can be configured to direct the light only toward a user's 150 hands when the user 150 is present so that the user 150 is unlikely to have the UV light directed at his or her eyes or body. Furthermore, the UV light source 116 can be configured to direct the UV light at the user's 150 hands the entire time the user 150 is present at the sanitizing system 100, or the UV light source 116 can be configured to direct the UV light at the user's 150 hands only during certain portions of the sanitizing process.

The sanitizing system 100 can include a proximity sensor 120 configured to detect the presence of a user 150. For example, the proximity sensor 120 can be configured to detect whether an object (e.g., a user's 150 hand) is within a predetermined distance from the proximity sensor 120. Alternatively, or in addition, the proximity sensor 120 can be configured to determine whether an object (e.g., a hand) has passed a predetermined threshold. The proximity sensor 120 can be configured to send a signal to the controller 130 when activated by the presence of a user 150. The proximity sensor 120 can be or include a thermal sensor, infrared sensor, ultrasonic sensor, acoustic sensor, image recognition sensor, pressure-sensitive sensor, radar, chemical sensor, inductive proximity sensor, capacitive sensor, photoelectric sensor, through-beam sensor, retro-reflective sensor, or any other type of sensor capable of sensing the presence of a user 150 and sending a signal to the controller 130 when activated.

The sanitizing system 100 can be configured to have a recess or aperture sized to receive a user's hands (or other object(s), as the case may be). In this configuration, a user 150 can approach the sanitizing system 100, and insert his or her hands into the recess or aperture to activate the proximity sensor 120 and begin sanitizing the user's 150 hands. Furthermore, the sanitizing system 100 can include one or more atomizing devices 112, 114 positioned to direct the fluid into the recess or aperture. For example, the sanitizing system 100 can include multiple atomizing devices positioned to direct the fluid toward the user's 150 hands from multiple directions and/or angles to ensure a sufficiently large portion of the users' 150 hands is covered with the fluid. Additionally, in this configuration, the sanitizing system 100 can include a blower 102 (with or without an air knife) configured to direct the air toward the user's 150 hands (or, for example, across the entry of the recess) such that any excess fluid is removed from the user's 150 hands as they are removed from the sanitizing device 100. Similarly, the sanitizing system 100 can include one or more UV light source 116 configured to direct the UV light toward the user's 150 hands from more than one direction while in the recess or aperture such that the user's 150 hands are sanitized on all sides at once. The UV light source 116 can be positioned within the recess (e.g., at an inner edge of the recess and/or at the entry of the recess), and the UV light source 116 can be configured to direct UV light radially inward toward the recess such that the UV light is directed away from the user's body.

The sanitizing system 100 can be controlled by a controller 130 configured to receive inputs from the various sensors (e.g., the proximity sensor 120 and the temperature sensor 122), determine appropriate actions based on the input, and control the output of the sanitizing system 100 to facilitate sanitizing a user's 150 hands. The controller 130 can have a memory 132 and a processor 134. The controller 130 can be or include a computing device configured to receive data, determine actions based on the received data, and output a control signal instructing one or more components of the system 100 to perform one or more actions. One of skill in the art will understand that the controller 130 can be installed in any location (e.g., within a housing of the sanitizing system 100; remotely from a housing of the sanitizing system 100, such as at a remote server), provided the controller 130 is in communication with at least some of the components of the system. Furthermore, the controller 130 can be configured to send and receive wireless or wired signals, and the signals can be analog or digital signals. The wireless signals can include Bluetooth™, BLE, WiFi™, ZigBee™, infrared, microwave radio, or any other type of wireless communication as may be appropriate for the particular application. The hard-wired signal can include any directly wired connection between the controller and the other components. For example, the controller 130 can have a hard-wired 24 VDC connection to the various components. Alternatively, the components can be powered directly from a power source and receive control instructions from the controller 130 via a digital connection. The digital connection can include a connection such as an Ethernet or a serial connection and can utilize any appropriate communication protocol for the application such as Modbus, fieldbus, PROFIBUS, SafetyBus p, Ethernet/IP, or any other appropriate communication protocol for the application. Furthermore, the controller 130 can utilize a combination of wireless, hard-wired, and analog or digital communication signals to communicate with and control the various components. One of skill in the art will appreciate that the above configurations are given merely as non-limiting examples and the actual configuration can vary depending on the application.

The controller 130 can include a memory 132 that can store a program and/or instructions associated with the functions and methods described herein and can include one or more processors 224 configured to execute the program and/or instructions. The memory 132 can include one or more suitable types of memory (e.g., volatile or non-volatile memory, random access memory (RAM), read only memory (ROM), programmable read-only memory (PROM), erasable programmable read-only memory (EPROM), electrically erasable programmable read-only memory (EEPROM), magnetic disks, optical disks, floppy disks, hard disks, removable cartridges, flash memory, a redundant array of independent disks (RAID), and the like) for storing files including the operating system, application programs (including, for example, a web browser application, a widget or gadget engine, and or other applications, as necessary), executable instructions and data. One, some, or all of the processing techniques described herein can be implemented as a combination of executable instructions and data within the memory.

The controller 130 can be configured to communicate with a user interface 136. The user interface 136 can be configured to display system information and instructions to the user 150. For example, the user interface 136 can be configured to instruct the user 150 what actions to perform such as where to position his or her hands to ensure the water and sanitizing solution are properly distributed, when to lather his or her hands, when to remove his or her hands from the sanitizing system 100 when the sanitizing process is completed, etc. The user interface 136 can also inform the user 150 of what actions the sanitizing system is performing so that the user 150 is aware of what is happening. Information provided by the user interface 136 can include information about what fluids are being dispensed, when the UV light is activated, when the air is being turned on to dry his or her hands, as system information such as a low battery indication, a low fluid indication, a clog in the system, etc. The user interface 136 can also be configured to receive inputs from a user 150 so that the user 150 can configure the sanitizing system 100 as desired. The user 150, for example, can input data to set the temperature of the air directed by the blower 102, the amount of time each component is configured to be operated during a sanitizing process, amounts of liquid to be directed toward the user's 150 hands, etc. The user interface 228 can be installed locally on or nearby the sanitizing system 100 or be a remote device such as a mobile device.

The various components of the sanitizing system 100 can be powered by a power supply. The power supply, for example, can include an electrical cord 140 configured to be plugged into an electrical outlet. The electrical outlet can provide power supplied from an electric utility grid or an alternative power source such as an electric generator or a solar power system. As another example, the power supply can be a battery 142 configured to provide power to the sanitizing system 100. As will be appreciated, sanitizing systems 100 configured to operate using battery power can be installed in locations where electrical outlets are unavailable, such as in various locations in a store, on a sidewalk, in a public park, at a school or place of business, or any other suitable location. As yet another example, the sanitizing system 100 can be configured to be powered by a photovoltaic power system such as a solar power system 144. The solar power system 144 can be used in conjunction with the battery 142 so that the solar power system 144 is capable of providing a charge to the battery 142. As will be appreciated, utilizing a solar power system 144 to power the sanitizing system 100 can provide the advantage of providing power to the sanitizing system 100 in locations where the sanitizing system 100 is capable of receiving adequate sunlight such as a park or other outdoor location.

The controller 130, by utilizing the memory 132 and the processor 134, can be configured to control the sanitizing system 100 to facilitate sanitizing the user's 150 hands. For example, the memory 132 can store instructions that, when executed by the processor 134, can cause the controller 130 and/or component(s) of the sanitizing system 100 to perform certain actions. The controller 130, for example, can receive proximity data from the proximity sensor 120. In response to determining that the proximity data indicates the presence of a user's 150 hand (or another object), the controller 130 can commence an application stage wherein the controller 130 can output instructions for the blower 102 to activate, instructions for the heating element 104 to activate, and/or instructions for water to be moved from the water reservoir 106 toward the user's 150 hand using any of the methods herein described (e.g., activating a pump, actuating a valve, etc.). The controller 130 can monitor the temperature of the heated air directed by the blower 102 based on temperature data received from one or more temperature sensors and can vary the output of the heating element 104 to ensure the air temperature (or liquid temperature) satisfies a target temperature. The controller 130 can provide instructions to deliver the water and the heated air for a predetermined amount of time, or to deliver a predetermined quantity of water, to allow a user 150 to rinse his or her hands. The controller 130 can cease directing the water from the water reservoir 106, and then provide instructions to begin directing a predetermined amount of the antibacterial agent from the antibacterial agent reservoir 108 using any of the methods herein described. Alternatively, the controller 130 can continue to provide heated air and water while directing the antibacterial agent from the antibacterial agent reservoir 108 so that antibacterial agent and water are delivered simultaneously.

After directing the antibacterial agent from the antibacterial agent reservoir 108 for a predetermined amount of time (or a predetermined quantity of antibacterial agent), the controller 130 can commence a waiting stage wherein the controller 130 can provide instructions to cease directing heated air, water, and antibacterial agent toward the user's 150 hands. The controller 130 can wait a predetermined amount of time before taking additional action, thereby permitting the user 150 can scrub his or her hands to kill and remove germs from his or her hands, without wasting water, antibacterial agent, or energy.

After the amount of time for washing has expired (i.e., the waiting stage), the controller 130 can commence a rinse stage wherein the controller 130 can reactivate the blower 102 and the heating element 104 and begin directing water from the water reservoir 106 to once again rinse the user's 150 hands to wash off the antibacterial solution. After the rinse stage the controller 130 can commence a drying stage wherein the controller 130 can cease directing water to the user's 150 hands and increase the speed of the blower 102 to direct heated air at a higher velocity (as compared to the air velocity associated with distributing the water/antibacterial agent) to dry the user's hands. If the sanitizing system 100 includes an air knife, the higher velocity created by the blower 102 in conjunction with the air knife can help to rapidly dry the user's hands.

At any time during the just described sanitizing process, the controller 130 can activate the UV light source 116 to provide additional sanitization of the user's 150 hands. The controller 130, for example, can activate the UV light source 116 as soon as the presence of a user 150 is detected and wait until the very end of the sanitizing process to deactivate the UV light source 116 so that UV light is able to provide a sanitizing benefit the entire time. Alternatively, the controller 130 can be configured to activate the UV light only once the user 150 has rinsed his or her hands the final time so that the user's 150 hands are further sanitized by the UV light only once the user 150 has already completed an initial sanitizing process by washing his or her hands using the sanitizing system 100. As one of skill in the art will appreciate, the controller 130 can be configured to activate the UV light source 116 for one or more predetermined lengths of times during various portions of the sanitizing process.

As described, the controller 130 can be configured to perform certain actions including waiting for predetermined lengths of time between actions. For example, the controller 130 can be configured to allow for five seconds for applying water (first part of application stage), five seconds for applying antibacterial solution (second part of application stage), twenty seconds of lathering (waiting stage), five seconds of rinsing (rinsing stage), and ten seconds of drying (drying stage). As one of skill in the art will appreciate, the predetermined amount of time for any portion of the sanitizing process can be varied depending on the particular application and the particular components utilized. For example, sanitizing systems utilizing an air knife may not need to dry the user's 150 hands for as long as systems without an air knife. As another example, some sanitizing system 100 configurations may require the controller 130 to allow for more time for the user 150 to rinse his or her hands. Furthermore, some sanitizing systems 100, for example, can be configured to direct the antibacterial agent faster than the five seconds just described. As yet another example, the controller 130 can be configured to deactivate any activated component if the controller 130 determines that a user 150 is no longer present as indicated by data received from the proximity sensor 120. These examples should not be construed as limiting as one of skill in the art will appreciate that the controller 130 can be configured to control the various elements of the sanitizing system 100 in any number of sequences and for any suitable amount of time to facilitate adequate sanitization for the particular application.

Although described in the context of being a sanitizing system 100 for sanitizing a user's 150 hands, the sanitizing system 100 can be modified for sanitizing other objects such as packages, countertops, door handles, elevator buttons, food, and other contaminated surfaces that individuals are likely to encounter. The sanitizing system 100, for example, can comprise a portable device configured to be used in much the same way as herein described for sanitizing a user's 150 hands. In this example, the user 150 could pick up the sanitizing system 100 and move it to a package or other surface to be disinfected and operate the sanitizing system 100 to disinfect the surface. Alternatively, the sanitizing system 100 can be a larger device intended for a user 150 to place an object inside of an enclosure and activate the sanitizing system 100 to disinfect the surface of the object. As one of skill in the art will appreciate, the disclosed technology can be modified to encompass these and other variations.

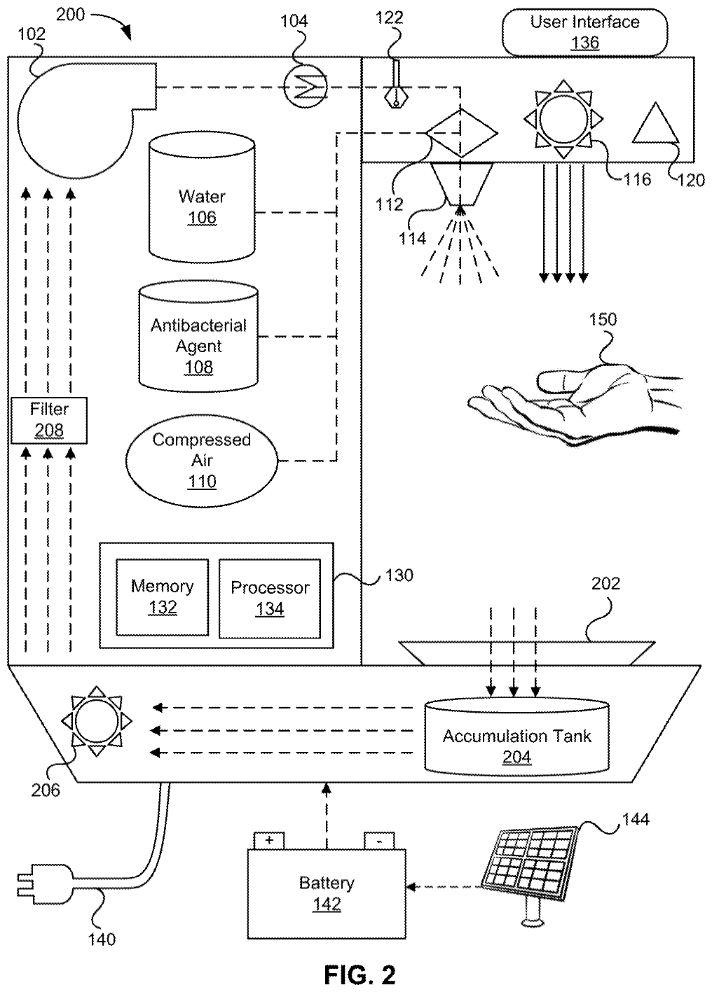

FIG. 2 depicts another example of the sanitizing system 200 that can be configured to collect any excess fluid that escapes past the user's 150 hands. This may be particularly advantageous for systems providing higher flow rates (e.g., greater than about 0.1 gpm, greater than about 0.25 gpm, greater than about 0.5 gpm) The sanitizing system 200 can include a vacuum inlet 202, an accumulation tank 204, a supplemental UV light source 206, and a filter 208. The vacuum inlet 202 can be configured to create a negative pressure so that air and any excess fluid that escapes past a user's 150 hands are directed into the vacuum inlet 202. As will be appreciated, the vacuum inlet 202 can provide a subsequent user a more pleasing user experience because the subsequent user is less likely to encounter germs that may be suspended in a mist left behind by a previous user. The negative pressure created at the vacuum inlet 202 can be created by a separate vacuum installed in the sanitizing system 200 or can be created by the inlet of the blower 102.

The accumulation tank 204 can be configured to collect excess fluid that has escaped past the user's 150 hands. The accumulation tank 204 can collect the fluid as it is collected by the vacuum inlet and directed through the sanitizing system 100. As will be appreciated, sanitizing systems that utilize ultra-low flows, such as the sanitizing system 100 or 200 herein described, are unlikely to produce much excess fluid therefore the accumulation tank 204 can be a relatively small size. Furthermore, the accumulation tank 204 may not need to be emptied as any collected fluid may evaporate quickly due to small amounts of fluid being collected. Alternatively, the accumulation tank 204 can be configured to have a drain that directs collected excess fluid out of the accumulation tank 204 and towards an existing drain (e.g., sink drain, floor drain, etc.), to the ground, or to some other location.

A supplemental UV light source 206 can be utilized to sanitize fluid droplets which may escape past the accumulation tank 204. The supplemental UV light source 206 can be the same as the UV light source 116 or the supplemental UV light source 206 can be a separate light source. If the supplemental UV light source 206 is separate from the UV light source 116, the supplemental UV light source 206 can be a different type of UV light source because the supplemental UV light source 206 can be operated without concern for impacting human health. For example, the supplemental UV light source 206 can be located inside of an enclosure such that a user 150 is protected from any UV light that could harm the user 150. Furthermore, the supplemental UV light source 206 can be configured to operate continuously or only when air and fluid is directed past the supplemental UV light source 206.

Alternatively, or in addition, the sanitizing system 200 can have a filter 208 configured to remove additional fluid that has escaped past the accumulation tank. The filter 208 can further reduce the likelihood that a fluid droplet carrying germs would be passed to a subsequent user after the user 150 has washed his or her hands.

As discussed previously in relation to the sanitizing system 100, the sanitizing system 200 can be configured to have a recess or aperture to receive the user's 150 hands. In this configuration, the vacuum inlet 202 can create a negative pressure within the recess or aperture such that air is directed through the recess or aperture from the inlet proximate the user's 150 hands and into the vacuum inlet 202. This configuration can help to ensure that any excess fluid is directed into the vacuum inlet 202 and not toward a subsequent user.

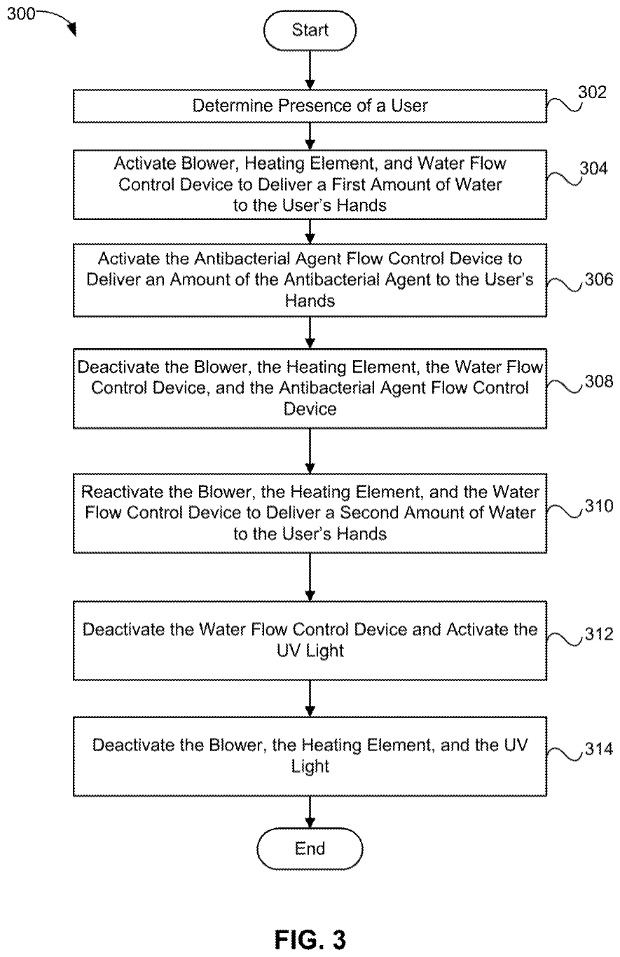

FIG. 3 is flowchart illustrating a method 300 of sanitizing hands using a sanitizing system, in accordance with an example of the disclosed technology. As described previously, the sanitizing system can be controlled by a controller configured to receive inputs and activate the various components based on determining the appropriate action. For example, the controller can execute a method 300 of sanitizing hands using the sanitizing system (e.g., a sanitizing process). The method 300 can include determining 302 the presence of a user's hands or other object (e.g., using a proximity sensor). Upon determining 302 the presence of an object, the method 300 can include activating 304 the blower, the heating element, and a water flow control device to direct heated air and a first amount of water toward the user's 150 hands to rinse the user's 150 hands. The method 300 can also include activating 306 the antibacterial agent flow control device to direct an amount of the antibacterial agent toward the user's hands. As will be appreciated, activating 306 the antibacterial agent flow control device can be performed subsequent to the water flow control device having directed the first amount of water toward the user's 150 hands, or the antibacterial agent flow control device and the water flow control device can be activated 304, 306 substantially simultaneously such that water and the antibacterial agent are provided at the same time. Alternatively, activating 304 the blower, the heating element, and the water flow control device can occur at a different time as compared to activating 306 the antibacterial agent flow control device but the activation times can be close enough that discharge of water and discharge of the antibacterial agent overlap at least momentarily.

The method can include deactivating 308 the blower, the heating element, the water flow control device, and the antibacterial agent flow control device to allow the user time to lather and the fluid mixture on his or her hands and/or wash his or hands using the fluid mixture. The method 300 can include reactivating 310 the blower, the heating element, and the water flow control device to direct a second amount of water toward the user's hands to rinse the user's hands. The method 300 can include deactivating 312 the water flow control device and activating the UV light to begin drying the user's hands and provide further disinfecting with the UV light. Alternatively, or additionally, the UV light can be activated at any point during the sanitizing process. The method can include deactivating 314 the blower, the heating element, and the UV light to end the sanitizing process. As will be appreciated, the method 300 just described can be varied in accordance with the various elements and examples described herein. That is, methods in accordance with the disclosed technology can include all or some of the steps described above and/or can include additional steps not expressly disclosed above. Further, methods in accordance with the disclosed technology can include some, but not all, of a particular step described above.

While the present disclosure has been described in connection with a plurality of exemplary aspects, as illustrated in the various figures and discussed above, it is understood that other similar aspects can be used, or modifications and additions can be made to the described aspects for performing the same function of the present disclosure without deviating therefrom. For example, in various aspects of the disclosure, methods and compositions were described according to aspects of the presently disclosed subject matter. But other equivalent methods or compositions to these described aspects are also contemplated by the teachings herein. Therefore, the present disclosure should not be limited to any single aspect, but rather construed in breadth and scope in accordance with the appended claims.

What is claimed is:

1. A hand-sanitizing system comprising:
a heating element for heating air;
a blower configured to direct, toward a user's hand, heated air heated by the heating element;
a first reservoir configured to hold a first fluid;
a second reservoir configured to hold a second fluid comprising a sanitizing agent;
an atomizing device configured to atomize at least one of the first fluid and the second fluid; and
a controller configured to activate the blower, the heating element, and the atomizing device to direct heated air, the first fluid, and the second fluid toward the user's hand.

2. The hand-sanitizing system of claim 1 further comprising an ultraviolet (UV) light configured to disinfect a surface of the user's hands, the UV light being configured to be activated by the controller.

3. The hand-sanitizing system of claim 2, wherein the UV light is a Far Ultraviolet-C (Far UV-C) light source.

4. The hand-sanitizing system of claim 1, wherein the blower is configured to create a venturi effect to direct at least one of the first fluid and the second fluid toward the user's hands.

5. The hand-sanitizing system of claim 1 further comprising a proximity sensor configured to detect a presence of the user's hand.

6. The hand-sanitizing system of claim 5, wherein the controller is further configured to activate the blower and the heating element in response to determining, based on proximity data received from the proximity sensor, that the user's hand is within a predetermined distance from the proximity sensor.

7. The hand-sanitizing system of claim 5, wherein the controller is further configured to deactivate the blower and the heating element if the presence of a user is no longer detected.

8. The hand-sanitizing system of claim 1, wherein the heating element is further configured to heat at least one of the first fluid and the second fluid.

9. The hand-sanitizing system of claim 1, wherein the atomizing device comprises an ultrasonic transducer.

10. The hand-sanitizing system of claim 1, wherein the atomizing device is configured to atomize at least one of the first fluid and the second fluid using compressed air.

11. The hand-sanitizing system of claim 1, wherein the sanitizing agent comprises at least one of electrolyzed water and an antibacterial solution.

12. The hand-sanitizing system of claim 1 further comprising a collection system configured to receive an overspray comprising at least some of the first fluid or at least some of the second fluid after the first fluid and the second fluid have been directed toward the user's hands.

13. The hand-sanitizing system of claim 12, wherein the collection system comprises a vacuum and an accumulation tank.

14. The hand-sanitizing system of claim 13, wherein the vacuum is provided by the blower.

15. The hand-sanitizing system of claim 1 further comprising a pump configured to transport at least some of the first fluid and at least some of the second fluid.

16. The hand-sanitizing system of claim 1 further comprising a first flow control valve configured to receive instructions from the controller and to permit flow of at least a portion of the first fluid.

17. The hand-sanitizing system of claim 1 further comprising a second flow control valve configured to receive instructions from the controller and to permit flow of at least a portion of the second fluid.

18. The hand-sanitizing system of claim 1, the sanitizing system further comprising a battery configured to power at least one of the heating element, the blower, the atomizing device, and the controller.

19. The hand-sanitizing system of claim 18 further comprising a solar power system configured to provide a charge to the battery.

20. A sanitizing system comprising:
a heating element for heating air;
a blower configured to direct, toward an object, heated air heated by the heating element;
a first reservoir configured to hold a first fluid;
a second reservoir configured to hold a second fluid comprising a sanitizing agent;
an atomizing device configured to atomize at least one of the first fluid and the second fluid; and
a controller configured to activate the blower, the heating element, and the atomizing device to direct heated air, the first fluid, and the second fluid toward the object.
